(19) United States
(12) Patent Application Publication
(10) Pub. No.: US 2018/0329713 A1
(43) Pub. Date: Nov. 15, 2018

(54) FITNESS SENSOR WITH LOW POWER ATTRIBUTES IN SENSOR HUB

(71) Applicant: Intel Corporation, Santa Clara, CA (US)

(72) Inventors: Ke Han, Shanghai (CN); Yong Hu, Shanghai (CN); Ke Ding, Shanghai (CN); Claire E. Jackoski, Chandler, AZ (US); Ray Kacelenga, Forest Grove, OR (US); Lama Nachman, Santa Clara, CA (US)

(73) Assignee: Intel Corporation, Santa Clara, CA (US)

(21) Appl. No.: 15/525,783

(22) PCT Filed: Dec. 10, 2014
(86) PCT No.: PCT/CN2014/093433

§ 371 Date: May 10, 2017

(51) Int. Cl. G06F 9/44; G06F 1/16; G06F 1/32; G06F 9/48

(52) U.S. Cl. CPC G06F 9/44; G06F 1/163; G06F 1/1694; A61B 5/11; G06F 9/48; A61B 2503/10; A61B 2560/0209; G06F 1/3215

(57) ABSTRACT

Systems and methods may provide for a fitness sensor that is located and operates in a sensor hub. The fitness sensor may link to a Bluetooth link controller, a communications hub and numerous environmental and physical sensors in a platform that is conducive to low power utilization. Awakening a host processor only when valid content-oriented sensor data is available may assist to reduce a footprint of power consumption and time spent in computer processing fitness models.

### CROSS-REFERENCE WITH RELATED APPLICATIONS

This application is a U.S. National Phase Patent Application which claims benefit to International Patent Application No. PCT/CN2014/093433 filed on Dec. 10, 2014.

### TECHNICAL FIELD

Embodiments generally relate to fitness sensors. More particularly, embodiments relate to the collection, computation, and extraction of fitness information directly in the firmware of a sensor hub.

### BACKGROUND

Fitness monitoring today may be divided into two categories—wearable device solutions and phone based application solutions. Wearable device solutions may be embedded solutions with efficient power, but may have insufficient information. The wearable device solutions may have limitations in size, weight, computation and power, which may be due to their portability. Phone based application solutions may provide sufficient data, but power may be inefficient. The applications in phone based solutions may poll data from sensors and location sources that will wake up a central processing unit (CPU) frequently and may lead to increased power consumption and/or reduced battery life.

### BRIEF DESCRIPTION OF THE DRAWINGS

The various advantages of the embodiments will become apparent to one skilled in the art by reading the following specification and appended claims, and by referencing the following drawings, in which:

FIG. 1 is a block diagram of an example of a fitness sensor computing system according to an embodiment;

FIG. 2 is a flowchart of an example of a method of operating a fitness sensor according to an embodiment;

FIG. 3 is a block diagram of an example of a closed loop architecture according to an embodiment;

FIG. 4 is a block diagram of an example of a fitness sensor in a sensor hub according to an embodiment; and

FIGS. 5A-5C are a flowchart of an example of a data sequence that may be followed by the closed loop architecture shown in FIG. 3.

### DESCRIPTION OF EMBODIMENTS

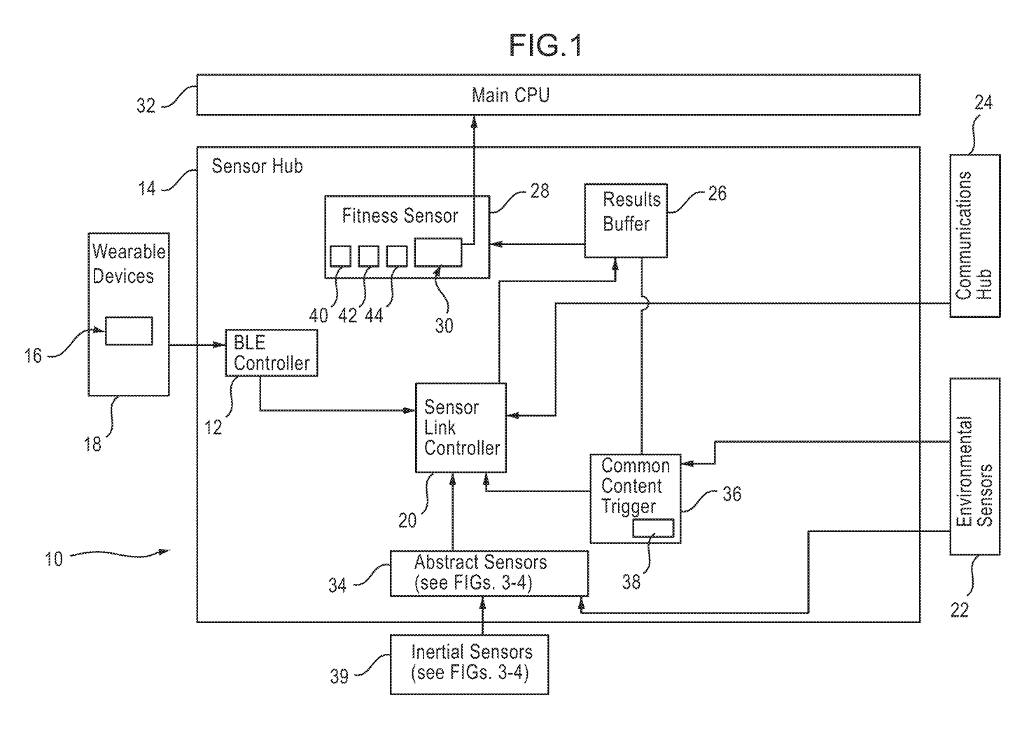

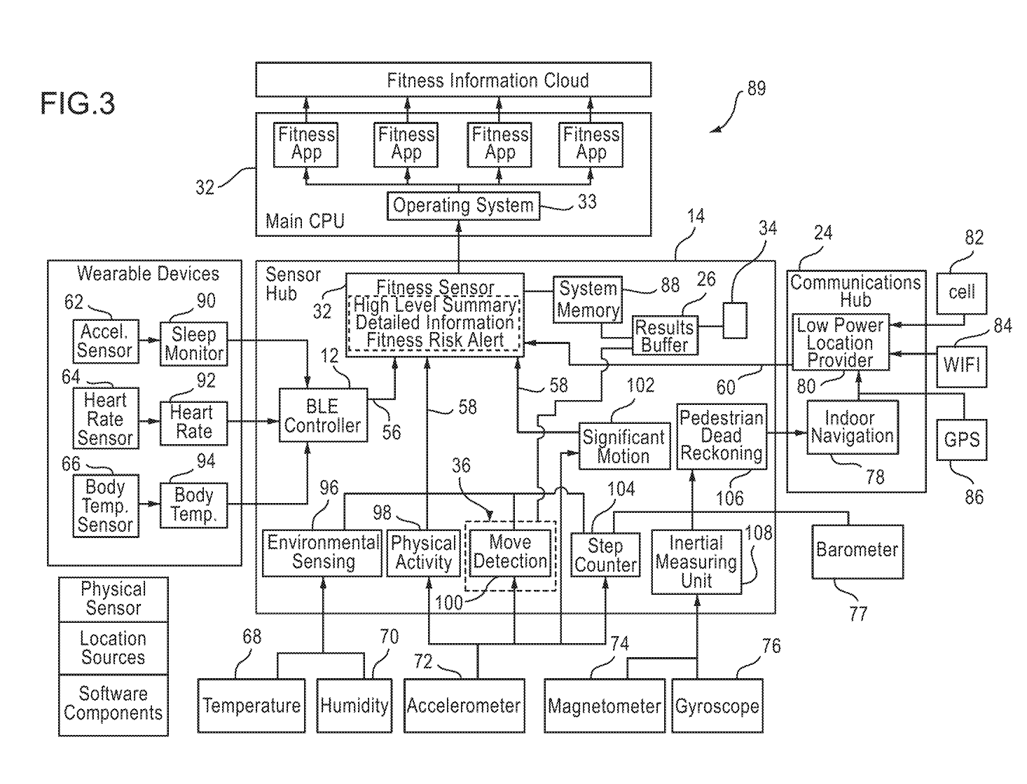

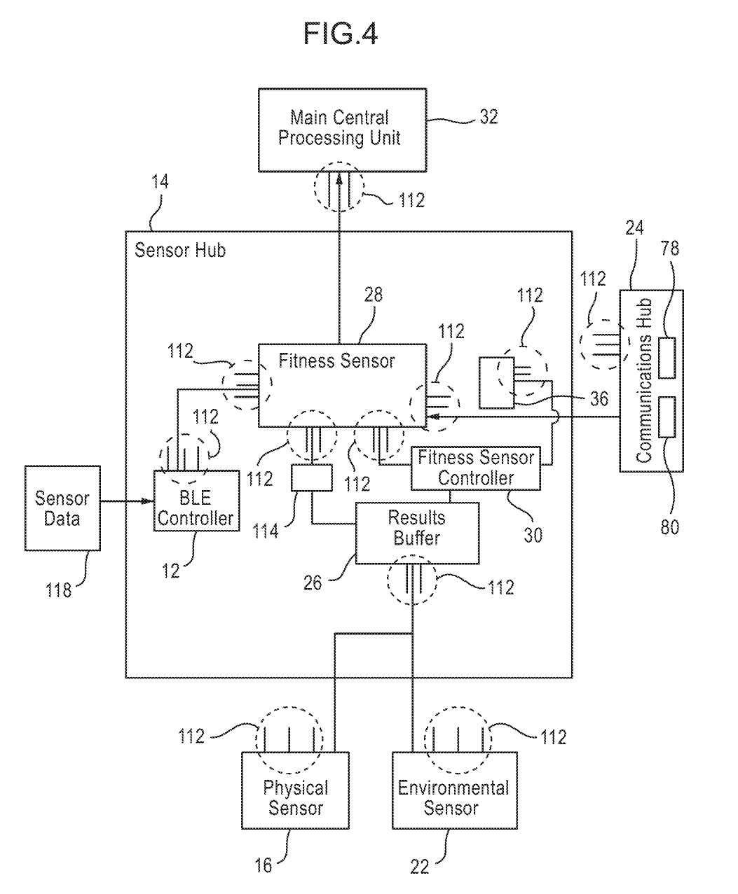

Turning now to FIG. 1, a fitness sensor computing system 10 is illustrated and including a sensor hub 14 having a fitness sensor 28. The fitness sensor computing system 10 may include a footprint platform (e.g., vendor-specific) that permits interconnections to various other associated platform devices with a low expenditure of power. The sensor hub 14 may operate at low power, have continuous sensing, and provide direct results. The illustrated sensor hub 14 by design is a low power engine. Thus, by offloading most of the computation bounded algorithm modules to the sensor hub 14, a reduction in processing use by a host processor 32 (e.g., central processing unit/CPU) running an operating system (see FIG. 3), albeit lower power consumption, can be achieved. A Bluetooth (e.g., Institute of Electrical and Electronics Engineers/IEEE 802.15.1-2005, Wireless Personal Area Networks) low energy (BLE) controller 12 in the sensor hub 14 may receive sensor data from physical sensors 16 that are positioned in wearable devices 18 (e.g., clothing, jewelry, eyewear, headwear). A sensor link controller 20 also located in the sensor hub 14 may receive the sensor data from the BLE controller 12, sensor data from abstract sensors 34 within the sensor hub 14 such as step counter, physical activity, etc.(see FIGS. 3-4) as well as data from a communications hub 24. Moreover, data from environmental sensors 22 and inertial sensors 39 may provide data input for the aforementioned activity sensor data activity within the sensor hub 14 (see FIGS. 3-4).

A results buffer 26 in the sensor hub 14 may store the sensor data received from the sensor link controller 20 in a compressed format. Additionally, a time stamp may be associated with the compressed data for future computational reference. The illustrated results buffer 26 stores the compressed data that will be processed to determine whether a sensor event has occurred. A sensor event may be associated with a high level summary such as calories, total steps, total distance, mean/min/max heart rate, mean/min/max body temperature, a portion of each physical activity, etc. The sensor event may also be associated with detailed information such as calories, temperature, heart rate, activity and steps over time, location change over time, etc. For example, the sensor event might include a fitness alert such as, for example, a sleep disorder, abnormal heartbeat, high/low fever, long time sedentary, lack of exercising, excessive exercising, etc. (see FIG. 3).

The results buffer 26 may transmit the sensor event to the fitness sensor 28, where it is further processed before transmission to a main central processing unit (CPU) 32 and for activation of an operating system. A common context trigger 36 in the sensor hub 14 may be connected to the results buffer 26 to configure the sampling rate of the sensor data. By using the common context trigger 36 in a low data sample rate only actual physical activity/terminal context/gesture may be captured and obscure abstract data may be discarded before processing. Thus, the illustrated computing system 10 eliminates power associated with processing unwanted, abstract sensor data. In this embodiment, a movement detection sensor 38 may be included in the common context trigger 36. The fitness sensor 28 may include a fitness sensor controller 30 to transmit the sensor events to the main CPU 32. As stated above, only the sensor event is transmitted to the main operating system (see FIG. 3), which may reduce power consumption and/or extend battery life. Because the host processor 32 is activated/awakened only when a sensor event is received, the main operating system (see FIG. 3) may remain dormant and cycle on only when actually needed for processing assignments.

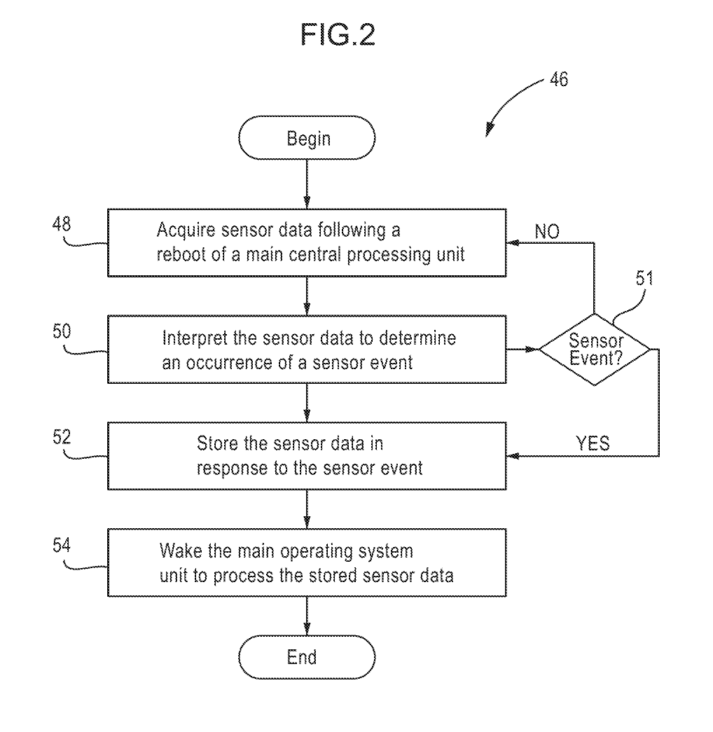

FIG. 2 describes a method 46 of operating a fitness sensor in a sensor hub. The method 46 may be implemented as a set of logic instructions stored in a machine- or computer-readable storage medium such as random access memory (RAM), read only memory (ROM), programmable ROM (PROM), firmware, flash memory, disk, etc., in configurable logic such as, for example, programmable logic arrays (PLAs), field programmable gate arrays (FPGAs), complex programmable logic devices (CPLDs), in fixed-functionality hardware logic using circuit technology such as, for example, application specific integrated circuit (ASIC), complementary metal oxide semiconductor (CMOS) or transistor-transistor logic (TTL) technology, or any combination thereof.

Following a reboot of the main operating system (see FIG. 3), sensor data may be acquired at illustrated processing block 48 from one or more of a BLE controller, physical sensors or the communications hub. The BLE controller may collect data from physical sensors such as, for example, an accelerometer sensor, a heart rate sensor and a body temperature sensor. The sensor data may also originate from other physical sensors such as a temperature sensor, a humidity sensor, a physical activity accelerometer sensor, a magnetometer sensor and a gyroscope sensor. The sensor data from the communications hub may originate from a cellular, WIFI (Wireless Fidelity, e.g., IEEE 802.11-2007, Wireless Local Area Network/LAN Medium Access Control (MAC) and Physical Layer (PHY) Specifications) and/or GPS (Global Positioning System) and include data from both indoor navigation and a low power location provider (see FIG. 3).

Block 50 may interpret the sensor data as a specific sensor event, save future processing time and identify concrete context material related to a fitness profile. As already discussed, the sensor events may be classified as high level summary data, detailed information data and fitness risk alert data. If there is a determination at block 51 that the sensor data acquired does not constitute a sensor event, then no further processing is commenced and the illustrated programming sequence returns to the acquisition of data phase at block 48. If, on the other hand, block 51 determines that the acquired sensor data does constitute a sensor event, then the sensor data may be stored at block 52 and classified as such for future transmission to a host processor for further processing. The stored sensor event may be transmitted to the main CPU at block 54 by the fitness sensor for processing by the operating system and further applied to requested fitness applications. The aforementioned transmission at block 54 of the sensor event to the main CPU may constitute the “waking” of the operating system into operation to initiate the processing sequence. The operating system may have been dormant until this time in a standby, power conservation mode awaiting an awakening by a transmission from the fitness sensor. Upon receiving the sensor event, the operating system may reboot and return to a standby mode.

Turning now to FIG. 3, a closed loop logic architecture 89 may be used to execute the fitness sensor computing system 10 such as described in FIG. 1 and the architecture may also implement one or more aspects of the method 46 (FIG. 2), or any combination thereof. In the illustrated example, the closed loop logic architecture 89 includes memory 88 that stores executable computer instructions. Sensor data may be acquired in the sensor hub 14 from physical sensors as acquired data 56, from environmental sensors as acquired data 58 using the BLE controller 12 and from communications sensors as acquired data 60 using the communications hub 24. The BLE controller 12 may obtain pertinent sensor data, for example, from a sleep accelerometer sensor 62 as sleep monitor data 90, from a heart rate sensor 64 as heart rate data 92 and from a body temperature sensor 66 as body temperature data 94. The data from the physical sensors 16 may include, for example, environmental sensing data 96 obtained from temperature sensor 68 and humidity sensor 70, physical activity data 98 obtained from physical activity accelerometer 72, motion/movement detection data 100 obtained from physical activity accelerometer sensor 72, significant motion/movement data 102 obtained from physical activity accelerometer sensor 72, step counter data 104 obtained from physical activity accelerometer 72, pedestrian dead reckoning data 106 obtained from gyroscope sensor 76, inertial measurement data 108 obtained from gyroscope sensor 76, and barometer data 77 obtained from barometer sensor 77. The sensor data from the communications hub 24 may be obtained from a cellular sensor 82 and designated as low power location provider data 80, from a WIFI sensor 84 and designated as also as low power location provider data 80, and a GPS sensor 86 and designated as indoor navigation data 78. The illustrated fitness sensor 28 interprets and stores the sensor data in a compressed state. A determination of the existence of a sensor event along with a time stamp may be applied to the compressed sensor data. The illustrated fitness sensor 28 categorizes the sensor event as high level summary data, detailed information data and fitness risk alert data. The movement detection data 100 may be used as the common context trigger 36 to sample the sensor data at a low sample rate and disregarding abstract data. The fitness sensor 28 transmits the sensor event to the main CPU 32 initiating the activation of the main operating system 33.

FIG. 4 describes a fitness sensor 28 positioned in a sensor hub 14. In the illustrated example, all connections are access points 112 specifically tailored for a fitness platform (not shown). The access points 112 for the BLE controller 12, communications hub 24, and physical and environmental sensors 16, 22 are configured to take advantage of the low power footprints (not shown) afforded by the designs of the aforementioned. By using the access points 112 for all communications to, and within the sensor hub 14, the fitness sensor 28 may take advantage of reduced power technologies inherently incorporated in the constituent components of the implemented fitness platform (not shown).

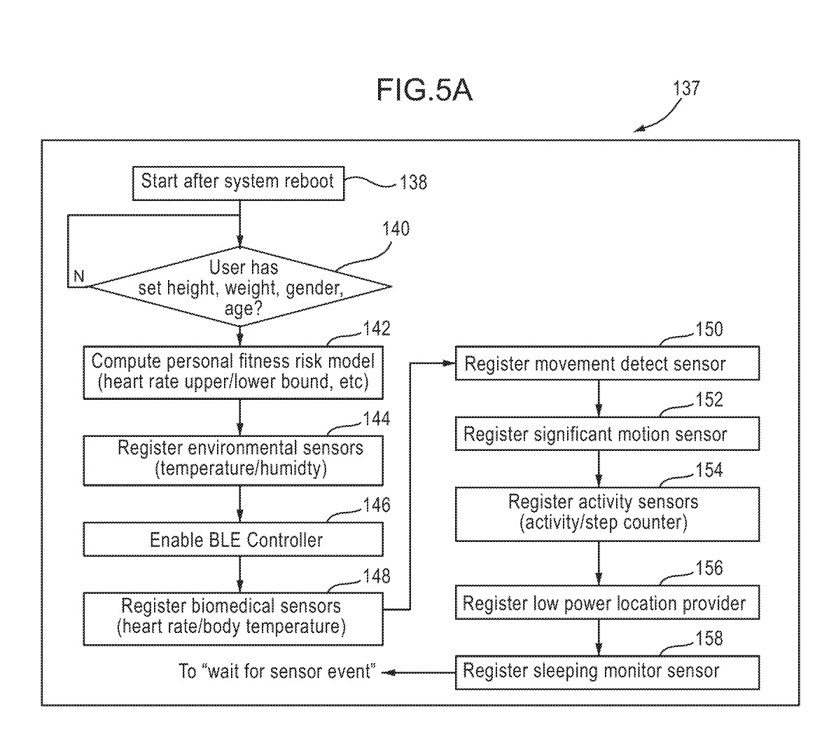

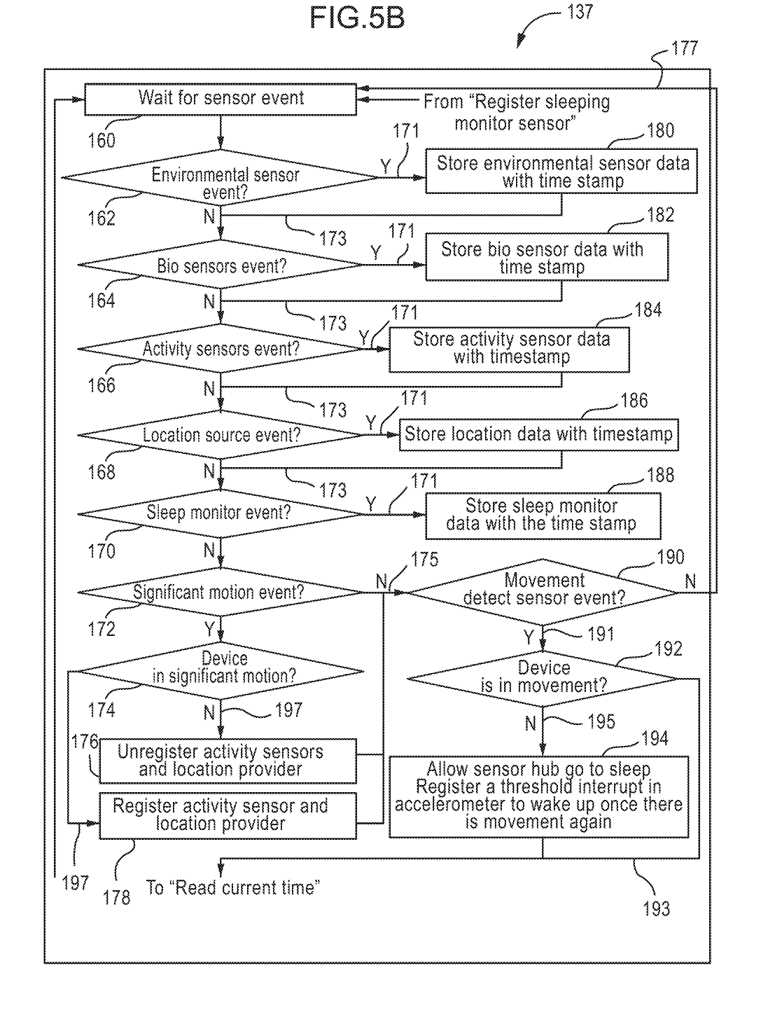

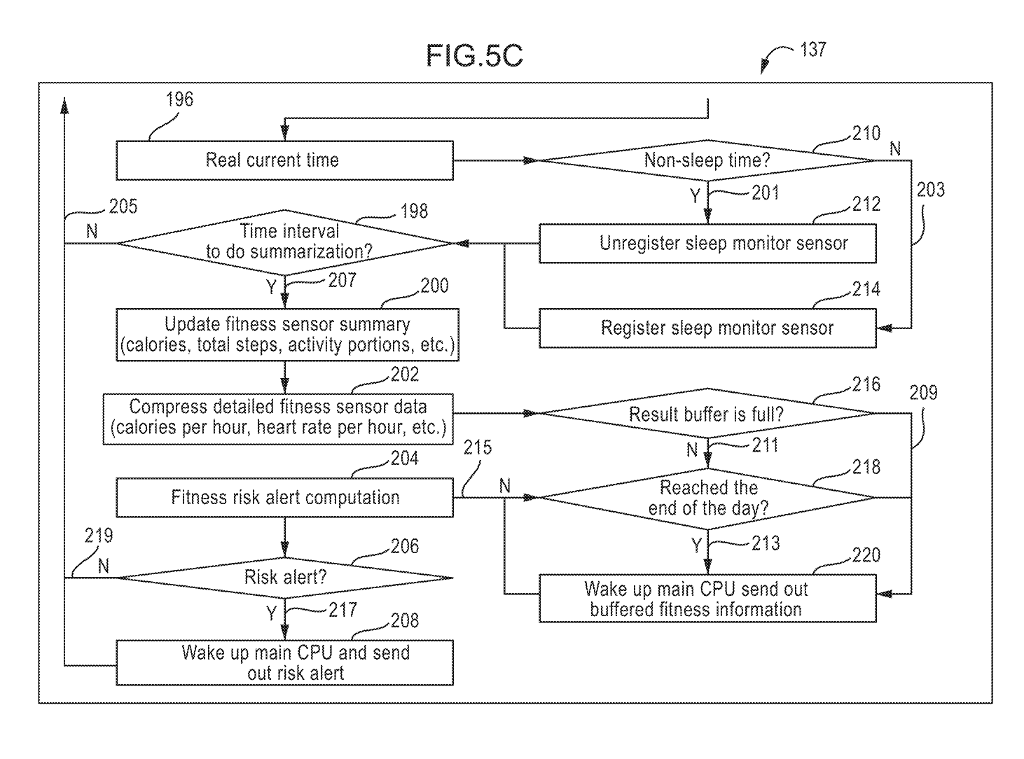

FIGS. 5A-5C describe a flowchart sequence 137 for a fitness sensor that may be followed to highlight the access points tailored to take advantage of a fitness platform with low power allocations. The illustrated sequence 137 begins with a reboot of the main operating system. The question may be presented as to whether personal physical data has been entered at illustrated block 140. A personal fitness risk model may then be computed at block 142. Environmental sensors are registered at block 144 and the data is transmitted at block 146 to the BLE controller. The BLE controller may register biomedical sensors such as, for example, heart rate sensors, body temperature sensors, etc., at block 148. The focus moves to register a movement detect sensor at illustrated block 150 and transitions even further to engage a significant motion sensor at block 152.

Additional data may be gathered from activity sensors at block 154, low power location provider at blocks 156, and sleeping monitor sensors at block 158. The sequence 137 may pause to determine the existence of a sensor event at block 160. The sequence 137 determines whether the sensor events are present for the following: an environmental sensors event at block 162, a bio sensors event at block 164, an activity sensors event at block 166, a location source event at block 168 and a sleep monitor event at block 170. The sequence sequentially queries for the existence of each event and where positive at block 171 may store the sensor data with a timestamp at blocks 180, 182, 184, 186, and 188. In the questioning for a significant motion event at block 172, a negative response at block 175 may lead to a determination of a movement detect sensor event at block 190.

If the movement detect sensor event is negative at block 177, the method may return to wait for a sequence event at block 160. If the movement detect sensor event at block 190 is positive at block 191 the question is asked whether the device is in movement at illustrated block 192. If it is determined that the device is in movement at block 193, the current time may be read at block 196. If it is determined that the device is not in movement at block 195, the sensor hub may be put in a “sleep” mode and a command to register a threshold interrupt in an accelerometer to “wake up” once there is movement again may be initiated at block 194. If the significant motion sensor event at block 172 is negative at block 197 the illustrated sequence 137 queries whether the device is in significant motion at block 174.

If the response is negative at block 197, the activity sensors and location provider may be unregistered at block 176. If the significant motion sensor events determination is positive at block 199 the activity sensors and location provider may be registered at block 178. Both the positive and negative responses may be sequenced to question whether there is a movement detect sensor event at block 190. After the current time is read at block 196, the illustrated sequence 137 queries whether it is non-sleep time at block 210. If the result is positive at block 201 the sleep monitor sensor is unregistered at block 212 and queries back to determine whether it is time to perform a time interval summarization at block 198. If the result is negative at block 203 the sequence 137 registers the sleep monitor sensor and questions whether it is time to perform a time interval summarization at block 198. A negative result at block 205 for the time interval summarization may return the method to a wait for a sensor event at block 160. A positive result at block 207 for the time interval summarization updates at illustrated block 200 the fitness sensory summary (calories, total steps, etc.) and compresses at block 202 detailed fitness sensor data (calories per hour, heart rate per hour, etc.).

The sequence 137 takes the compressed sensor data and may pose the question whether the result buffer is full at block 216. If the result buffer is full at block 209, the main operating system is activated to send out buffered fitness information at block 220 and moves to a fitness risk alert computation at block 204. If the result buffer is not full at block 211, the question is asked whether it is the end of the day at block 218. If it is the end of the day at block 213, the main operating system is activated at block 220 as above. If it is not the end of the day at block 215, the illustrated sequence 137 moves to a fitness risk alert computation at block 204. The sequence 137 may question whether the data warrants a risk alert at block 206. If a risk alert is warranted at block 217, the main operating system may be activated to send out a risk alert at block 208. If a risk alert is not warranted at block 219, the illustrated sequence 137 returns to wait for a sensor event at block 160.

The illustrated sequence 137 therefore illustrates that if no movement is detected, both the sensor hub and location source are put in a standby mode. Only related sensors are accessed if needed such as open the sleep monitor at night. In this illustration, the significant motion sensor is used to open/close activity/step sensor automatically. The main operating system is only activated, “awakened”, when the data buffer is full, or when the time has reached the end of the day. The aforementioned conservation measures may all present a savings in power consumption in the platform.

### ADDITIONAL NOTES AND EXAMPLES

Example 1 may include a computing system comprising, a host processor, a communications hub and a sensor hub including a Bluetooth link controller to receive first sensor data from physical sensors in one or more wearable devices, a sensor link controller to receive second sensor data from the Bluetooth link controller, the communications hub and one or more abstract sensors, a results buffer to store the sensor data from the sensor link controller in a compressed data format with a time stamp and hold the sensor data for future processing to determine whether the compressed data constitutes a sensor event, and a fitness sensor controller to awaken the host processor and transmit the sensor event data to the host to begin processing the stored sensor data.

Example 2 may include the computing system of Example 1, wherein a common context trigger in the sensor hub is to assist the results buffer to determine a sample rate for the sensor data.

Example 3 may include computing system of Example 1, wherein a common context trigger is to assist the results buffer to receive non abstract sensor data from the physical and environmental sensors.

Example 4 may include the computing system of Example 3, wherein the common context trigger includes include a movement detection sensor.

Example 5 may include the computing system of Example 1, wherein the fitness sensor controller transmits the sensor event data in one or more data forms as a high level summary, detailed information, or a fitness risk alert.

Example 6 may include the computing system of any one of Examples 1 to 5, wherein the computing system includes include a vendor-specific platform.

Example 7 may include a method of operating a sensor hub, comprising acquiring sensor data of a host processor, interpreting the sensor data to determine an occurrence of a sensor event, storing the sensor data in response to the sensor event, and queuing the host processor to process the stored sensor data.

Example 8 may include the method of Example 7, wherein the sensor data is acquired from one or more of a Bluetooth controller, physical sensors or a communications hub.

Example 9 may include the method of Example 8, wherein the sensor data from the Bluetooth controller includes data from one or more of an accelerometer sensor, a heart rate sensor or a body temperature sensor.

Example 10 may include the method of Example 8, wherein the sensor data from the physical sensors includes data from one or more of a temperature sensor, a humidity sensor, a physical activity accelerometer sensor, a magnetometer sensor or a gyroscope sensor.

Example 11 may include the method of Example 8, wherein the sensor data from the communications hub includes indoor navigation data from a low power location provider including one or more of a cellular, WIFI or GPS location source.

Example 12 may include the method of any one of Examples 7 to 11, wherein the sensor data from the sensor event is stored with a time stamp as compressed data including one or more of a high level summary, detailed information, or a fitness risk alert.

Example 13 may include at least one non-transitory computer readable storage medium comprising a set of instructions which, when executed by a computing system, cause the computing system to acquire sensor data of a host processor, interpret the sensor data to determine an occurrence of a sensor event, store the sensor data in response to the sensor event, and queue the host processor to process the stored sensor data.

Example 14 may include the at least one non-transitory computer readable storage medium of Example 13, wherein the instructions, when executed, cause a computing system to acquire sensor data from at least one or more of a Bluetooth controller, a communications hub or physical sensors.

Example 15 may include the at least one non-transitory computer readable storage medium of Example 14, wherein the sensor data from the Bluetooth controller includes sleep monitor data, heart rate data and body temperature data, and wherein the sensor data from the communications hub includes low power location provider data and indoor navigation data, and further wherein the sensor data from the physical sensors includes one or more of environmental sensing data, physical activity data, motion detection data, significant motion data, step counter data, pedestrian dead reckoning data, barometer data, or inertial measurement data.

Example 16 may include the at least one non-transitory computer readable storage medium of Example 13, wherein the compressed data format includes a time stamped sensor event with high level summary data, detailed information data, and fitness risk alert data.

Example 17 may include the at least one non-transitory computer readable storage medium of Example 15, wherein the motion detection data includes a common context trigger having the acquired sensor data with a low sample rate.

Example 18 may include the at least one non-transitory computer readable storage medium of any one of Examples 13 to 16, wherein a common context trigger within the sensor hub is to determine a sensor data sample time interval before transmitting the compressed format sensor data to the main central processing unit.

Example 19 may include a fitness sensor hub, comprising, access points to a Bluetooth controller, a communications hub, and physical and environmental sensors, a results buffer to gather and store sensor data transmitted to the access points, and a fitness sensor controller to interpret and transmit the sensor data from the results buffer to a main operating system.

Example 20 may include the fitness sensor hub of Example 19, wherein the Bluetooth controller is positioned in the sensor hub and, using the access points, receives sensor data including sleep monitor, heart rate and body temperature.

Example 21 may include the fitness sensor hub of Example 19, wherein the results buffer processes the sensor data as a sensor event and stores and categorizes the sensor event as one or more of high level summary, detailed information, or fitness risk alert.

Example 22 may include the fitness sensor hub of Example 19, wherein the sensor data from the communications hub is to include indoor navigation data from a low power location provider including one or more of a cellular, WIFI or GPS location source.

Example 23 may include the fitness sensor of Example 19, wherein the physical and environmental sensors include temperature, humidity, accelerometer, magnetometer, gyroscope and barometer.

Example 24 may include the fitness sensor hub of any one of Examples 19 to 23, wherein the fitness sensor controller, using the access points, interfaces with a common context trigger to regulate the sensor data content, and to regulate the sensor data sample rate before transmission of the sensor data to the main operating system.

Example 25 may include a fitness sensor hub comprising means for performing the method of any of Examples 7 to 12, in any combination or sub-combination.

Embodiments are applicable for use with all types of semiconductor integrated circuit (IC) chips. Examples of these IC chips include but are not limited to processors, controllers, chipset components, programmable logic arrays, memory chips, network chips, systems on chips (SoCs), SSD/NAND controller ASICs, and the like. In addition, in some of the drawings, signal conductor lines are represented with lines. Some may be different, to indicate more constituent signal paths, have a number label, to indicate a number of constituent signal paths, and/or have arrows at one or more ends, to indicate primary information flow direction. This, however should not be construed in a limiting manner. Rather, such added detail may be used in connection with one or more exemplary embodiments to facilitate easier understanding of a circuit. Any represented signal lines, whether or not having additional information, may actually comprise one or more signals that may travel in multiple directions and may be implemented with any suitable type of signal scheme, e.g., digital or analog lines implemented with differential pairs, optical fiber lines, and/or single-ended lines.

Example sizes/models/values/ranges may have been given, although embodiments are not limited to the same. As manufacturing techniques (e.g. photolithography) mature over time, it is expected that devices of smaller size could be manufactured. In addition, well known power/ground connections to IC chips and other components may or may not be shown within the figures, for simplicity of illustration and discussion, and so as not to obscure certain aspects of the embodiments. Further, arrangements may be shown in block diagram form in order to avoid obscuring embodiments, and also in view of the fact that specifics with respect to implementation of such block diagram arrangements are highly dependent upon the platform within which the embodiment is to be implemented, i.e., such specifics should be well within purview of one skilled in the art. Where specific details (e.g., circuits) are set forth in order to describe example embodiments, it should be apparent to one skilled in the art that embodiments can be practiced without, or with variation of, these specific details. The description is thus to be regarded as illustrative instead of limiting.

The term “coupled” may be used herein to refer to any type of relationship, direct or indirect, between the components in question, and may apply to electrical, mechanical, fluid, optical, electromagnetic, electromechanical or other connections. In addition, the terms “first”, “second”, etc. may be used herein only to facilitate discussion, and carry no particular temporal or chronological significance unless otherwise indicated.

As used in this application and in the claims, a list of items joined by the term “one or more of” may mean any combination of the listed terms. For example, the phrases “one or more of A, B or C” may mean A, B, C; A and B; A and C; B and C; or A, B and C.

Those skilled in the art will appreciate from the foregoing description that the broad techniques of the embodiments can be implemented in a variety of forms. Therefore, while the embodiments have been described in connection with particular examples thereof, the true scope of the embodiments should not be so limited since other modifications will become apparent to the skilled practitioner upon a study of the drawings, specification, and the following claims.

We claim:

1. A computing system comprising:
a host processor;
a communications hub; and
a sensor hub including,
a Bluetooth link controller to receive first sensor data from physical sensors in one or more wearable devices,
a sensor link controller to receive second sensor data from the Bluetooth link controller, one or more abstract sensors and the communications hub,
a results buffer to store the sensor data from the sensor link controller in a compressed data format with a time stamp, and hold the sensor data for future processing to determine whether the compressed data constitutes a sensor event, and
a fitness sensor controller to awaken the host processor and transmit the sensor event data to the host processor to begin processing the stored sensor data.

2. The computing system of claim 1, wherein a common context trigger in the sensor hub is to assist the results buffer to determine a sample rate for the sensor data.

3. The computing system of claim 1, wherein a common context trigger is to assist the results buffer to receive non abstract sensor data from the physical and environmental sensors.

4. The computing system of claim 3, wherein the common context trigger includes a movement detection sensor.

5. The computing system of claim 1, wherein the fitness sensor controller transmits the sensor event data in one or more data forms as a high level summary, detailed information, or a fitness risk alert.

6. The computing system of claim 1, wherein the computing system includes a vendor-specific footprint platform.

7. A method of operating a sensor hub, comprising:
acquiring sensor data of a host processor;
interpreting the sensor data to determine an occurrence of a sensor event;
storing the sensor data in response to the sensor event; and
queuing the host processor to process the stored sensor data.

8. The method of claim 7, wherein the sensor data is acquired from one or more of a Bluetooth controller, physical sensors or a communications hub.

9. The method of claim 8, wherein the sensor data from the Bluetooth controller includes data from one or more of an accelerometer sensor, a heart rate sensor or a body temperature sensor.

10. The method of claim 8, wherein the sensor data from the physical sensors includes data from one or more of a temperature sensor, a humidity sensor, a physical activity accelerometer sensor, a magnetometer sensor or a gyroscope sensor.

11. The method of claim 8, wherein the sensor data from the communications hub includes indoor navigation data from a low power location provider including one or more of a cellular, WIFI or GPS location source.

12. The method of claim 7, wherein the sensor data from the sensor event is stored with a time stamp as compressed data including one or more of a high level summary data, detailed information, or a fitness risk alert.

13. At least one non-transitory computer readable storage medium comprising a set of instructions which, when executed by a computing system, cause the computing system to:
acquire sensor data of a host processor;
interpret the sensor data to determine an occurrence of a sensor event;
store the sensor data in response to the sensor event; and
queue the host processor to process the stored sensor data.

14. The at least one non-transitory computer readable storage medium of claim 13, wherein the instructions, when executed, cause a computing system to acquire sensor data from at least one or more of a Bluetooth controller, a communications hub or physical sensors.

15. The at least one non-transitory computer readable storage medium of claim 14, wherein the sensor data from the Bluetooth controller includes sleep monitor data, heart rate data and body temperature data, and wherein the sensor data from the communications hub includes low power location provider data and indoor navigation data, and further wherein the sensor data from the physical sensors is to include one or more of environmental sensing data, physical activity data, motion detection data, significant motion data, step counter data, pedestrian dead reckoning data, barometer data, or inertial measurement data.

16. The at least one non-transitory computer readable storage medium of claim 13, wherein the compressed data format is to include a time stamped sensor event with one or more of a high level summary, detailed information, or a fitness risk alert.

17. The at least one non-transitory computer readable storage medium of claim 15, wherein the motion detection data is to include a common context trigger having the acquired sensor data with a low sample rate.

18. The at least one non-transitory computer readable storage medium of claim 13, wherein a common context trigger within the sensor hub is to determine a sensor data sample time interval before transmitting the compressed format sensor data to the main central processing unit.

19. A fitness sensor hub, comprising:
access points to a Bluetooth controller, a communications hub, and physical and environmental sensors;
a results buffer to gather and store sensor data transmitted to the access points; and
a fitness sensor controller to interpret and transmit the sensor data from the results buffer to a main operating system.

20. The fitness sensor hub of claim 19, wherein the Bluetooth controller is positioned in the sensor hub and, using the access points, receives sensor data including sleep monitor data, heart rate and body temperature.

21. The fitness sensor hub of claim 19, wherein the results buffer processes the sensor data as a sensor event and stores and categorizes the sensor event as one or more of high level summary, detailed information, or fitness risk alert.

22. The fitness sensor hub of claim 19, wherein the sensor data from the communications hub is to include indoor navigation data from a low power location provider including one or more of a cellular, WIFI or GPS location source.

23. The fitness sensor of claim 19, wherein the physical and environmental sensors include temperature, humidity, accelerometer, magnetometer, gyroscope and barometer.

24. The fitness sensor hub of claim 19, wherein the fitness sensor controller, using the access points, interfaces with a common context trigger is to regulate the sensor data content, and a common context trigger to regulate the sensor data sample rate before transmission of the sensor data to the main operating system.
